(19) United States
(12) Patent Application Publication
(10) Pub. No.: US 2026/0035587 A1
(43) Pub. Date: Feb. 5, 2026

(54) METHODS OF IMPROVING EMISSION OUTPUT OF COATED ENERGY MODULATION AGENTS AND COMPOSITIONS PRODUCED THEREBY

(71) Applicant: Immunolight, LLC, Detroit, MI (US)

(72) Inventors: Zakaryae FATHI, Raleigh, NC (US); Wayne F. BEYER, Jr., Ivanhoe, VA (US)

(73) Assignee: Immunolight, LLC, Detroit, MI (US)

(21) Appl. No.: 19/099,909

(22) PCT Filed: Aug. 17, 2023
(86) PCT No.: PCT/US2023/072377

§ 371 Date: Jan. 30, 2025

Related U.S. Application Data
(60) Provisional application No. 63/373,813, filed on Aug. 29, 2022.

(51) Int. Cl. C09D 101/08; C09D 1/00; C09D 5/00

(52) U.S. Cl. CPC C09D 101/08; C09D 1/00; C09D 5/006

(57) ABSTRACT

A method for increasing emission output from an energy modulation agent, involving:
drying the energy modulation agent in particulate form to reduce moisture level of the energy modulation agent by at least 25% to provide a dried energy modulation agent; and
coating the dried energy modulation agent with a coating having high transmissibility at a wavelength of primary emission from the energy modulation agent upon excitation,
and energy modulation agents prepared thereby.

### CROSS REFERENCE TO RELATED APPLICATIONS

This application claims priority to U.S. Provisional Ser. No. 63/373,813, filed Aug. 29, 2022, the entire contents of which are hereby incorporated herein by reference.

### BACKGROUND OF THE INVENTION

### Field of the Invention

The present invention relates to methods for improving the emission output, or light output, of a coated energy modulation agent, and the compositions produced by the method.

### Description of the Related Art

Light modulation from a deeply penetrating radiation like X-ray opens the possibility for activating bio-therapeutic agents of various kinds within mammalian bodies. Other possibilities include the activation of photo-catalysts in mediums for cross-linking reactions in polymeric chains and polymer based adhesives. These examples are but two examples of a number of possibilities that can be more generally described as the use of a conversion material to convert an initiating radiation that is deeply penetrating to another useful radiation possessing the capability of promoting photo-based chemical reactions. The photo-chemistry is driven inside mediums of far ranging kinds including organic, inorganic or composited from organic and inorganic materials.

The photo-activation with no line of site required can be done in-vivo and ex-vivo such as those carried out in cell cultures. In turn, the photo activation of select bio-therapeutic agent, and conceivably more than one agent at a time, can lead to the onset of a desirable chemical reaction, or a cascade of reactions, that in turn lead to a beneficial therapeutic outcome. As an example, the binding of psoralen to DNA through the formation of monoadducts is well known to engender an immune response if done properly. An in-depth treatise of the subject is available in the open literature. Psoralen under the correct photo-catalytic light gains the aptitude to bind to DNA. Psoralen has been reported to react to other sites that have a suitable reactivity including and not limited to cell walls. If this reaction is of the correct kind, as is the case for psoralen-DNA monoadducts formation, the binding leads to a programmable cell death referred to as Apoptosis. Such programmable cell death, if accomplished over a sufficiently large cell population. can signal the body to mount an immune response enabling target specific cell kill throughout the body. Such immune response is of the upmost importance for various medical treatments including cancer cure.

The cascade of events described above has at its source the modulation of electromagnetic energy from the X-ray to the UV energy using phosphors in the presence of bio-therapeutic agents; these methods and the like, have been thoroughly described in various patents and patent applications such as those listed in the cross-reference section above.

In particular, in U.S. Ser. No. 11/935,655, entitled “METHODS AND SYSTEMS FOR TREATING CELL PROLIFERATION DISORDERS,” the use of a phosphorescent emitting source was described with the advantage of phosphorescent emitting molecules or other source may be electroactivated or photoactivated prior to insertion into the tumor either by systemic administration or direct insertion into the region of the tumor. Phosphorescent materials have longer relaxation times than fluorescent materials. Energy emission is delayed or prolonged from a fraction of a second to several hours. Otherwise, the energy emitted during phosphorescent relaxation is not otherwise different than fluorescence, and the range of wavelengths may be selected by choosing a particular phosphor.

In particular, in U.S. Ser. No. 12/401,478, entitled “PLASMONIC ASSISTED SYSTEMS AND METHODS FOR INTERIOR ENERGY-ACTIVATION FROM AN EXTERIOR SOURCE,” the use of phosphorescent materials as energy modulation agents was described. The '478 application details a number of modulation agents some having a very short energy retention time (on the order of fs-ns. e.g. fluorescent molecules) whereas others having a very long half-life (on the order of seconds to hours, e.g. luminescent inorganic molecules or phosphorescent molecules). Specific types of energy modulation agents described in the '478 application included Y₂O₃; ZnS; ZnSe; MgS; CaS; Mn, Er ZnSe; Mn, Er MgS; Mn, Er CaS; Mn, Er ZnS; Mn,Yb ZnSe; Mn,Yb MgS; Mn, Yb CaS; Mn,Yb ZnS:Tb³⁺, Er³⁺; ZnS:Tb³⁺; Y₂O₃:Tb³⁺; Y₂O₃:Tb³⁺, Er3⁺; ZnS:Mn²⁺; ZnS:Mn,Er³⁺.

Presently, light (i.e., electromagnetic radiation from the radio frequency through the visible to the x-ray and gamma ray wavelength range) activated processing is also used in a number of industrial processes ranging from photoresist curing, to on-demand ozone production, to sterilization, to the promotion of polymer cross-linking activation (e.g. in adhesive and surface coatings) and others. Today, light activated processing is seen in these areas to have distinct advantages over more conventional approaches. For example. conventional sterilization by steam autoclaving or in food processing by pasteurization may unsuitably overheat the medium to be sterilized. As such, light activated curable coatings are one of the fastest growing sectors in the coatings industry. In recent years, this technology has made inroads into a number of market segments like fiber optics, optical and pressure-sensitive adhesives, and automotive applications like cured topcoats, and curable powder coatings. The driving force of this development is mostly the quest for an increase in productivity of the coating and curing process, as conventional non light activated adhesive and surface coatings typically require 1) the elimination of solvents from the adhesive and surface coatings to produce a cure and 2) a time/temperature cure which adds delay and costs to the manufacturing process.

Moreover, the use of solvent based products in adhesive and surface coatings applications is becoming increasingly unattractive because of rising energy costs and stringent regulation of solvent emissions into the atmosphere. Optimum energy savings as well as beneficial ecological considerations are both served by radiation curable adhesive and surface coating compositions. Radiation curable polymer cross-linking systems have been developed to eliminate the need for high oven temperatures and to eliminate the need for expensive solvent recovery systems. In those systems, light irradiation initiates free-radical cross-linking in the presence of common photosensitizers.

However, in the adhesive and surface coating applications and in many of the other applications listed above, the light-activated processing is limited due to the penetration depth of light into the processed medium. For example, in water sterilization, ultraviolet light sources are coupled with agitation and stirring mechanisms in order to ensure that any bacteria in the water medium will be exposed to the UV light. In light-activated adhesive and surface coating processing, the primary limitation is that the material to be cured must be directly exposed to the light, both in type (wavelength or spectral distribution) and intensity. In adhesive and surface coating applications, any “shaded” area will require a secondary cure mechanism, increasing cure time over the non-shaded areas and further delaying cure time due to the existent of a sealed skin through which subsequent curing must proceed (i.e., referred to as a cocoon effect).

In each of the above noted industries and treatments, an important factor is the amount of light needed to cause the light activation. When such light is being generated by the use of an energy modulation agent (in vivo in a patient or without line of sight in an industrial activation) which converts applied energy into an emitted energy of the desired wavelength, the amount of emission output from the energy modulation agent becomes an important factor to success. In certain instances, when the energy modulation agent must be coated to ensure biocompatibility

### SUMMARY OF THE INVENTION

Accordingly, one object of the present invention is to provide a method to improve or increase the emission output of an energy modulation having a coating with high transmissibility at the emission wavelength.

A further object of the present invention is to provide such a method for an energy modulation agent having a diamond or diamond-like carbon coating.

A further object of the present invention is to provide energy modulation agents prepared by the method of the invention which have significantly improved emission output.

These and other objects of the invention, alone or in combinations, have been satisfied by the discovery of a method for increasing emission output from an energy modulation agent, comprising drying the energy modulation agent in particulate form at a temperature and/or pressure sufficient to reduce moisture level of the energy modulation agent by at least 25% to provide a dried energy modulation agent: and coating the dried energy modulation agent with a coating having high transmissibility at a wavelength of primary emission from the energy modulation agent upon excitation.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a graphical representation of the transmission differences between the two slides having differing thicknesses of DLC coating over a range of wavelengths from about 190 nm to about 380 nm

FIG. 2 is a graphical representation of the percentage improvement in transmission at each wavelength over that range for the thinner coating (consistently higher transmission).

FIG. 3 is a graphical representation of the emission intensity of phosphors that were not dried, then coated with the 86 nm DLC coating.

FIG. 4 is a graphical representation of the emission intensity of phosphors that were dried then coated with the thinner 65 nm DLC coating

FIG. 5 is a graphical representation comparing the average UV emission of various phosphors coated using DLC at different thicknesses contrasted between phosphors dried under negative pressure (see data points marked “x”) and phosphors that were not dried (see data points marked “⋅”).

### DETAILED DESCRIPTION OF THE PREFERRED EMBODIMENTS

The present invention relates to a method for increasing emission output from an energy modulation agent, comprising:
drying the energy modulation agent in particulate form to reduce moisture level of the energy modulation agent by at least 25% to provide a dried energy modulation agent; and
coating the dried energy modulation agent with a coating having high transmissibility at a wavelength of primary emission from the energy modulation agent upon excitation.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the terms “at” or “about,” as used herein when referring to a measurable value or metric is meant to encompass variations of 20%, 10%, 5%, 1%, 0.5%, or even 0.1% of the specified amount, for example a specified ratio, a specified thickness, a specified phosphor size, or a specified water contact angle. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

### Energy Modulation Agent

As used herein, an “energy modulation agent” refers to an agent that is capable of receiving an energy input from a source and then re-emitting a different energy to a receiving target. Energy transfer among molecules may occur in a number of ways. The form of energy may be electronic, thermal, vibronic, electromagnetic, kinetic, or chemical in nature. Energy may be transferred from one molecule to another (intermolecular transfer) or from one part of a molecule to another part of the same molecule (intramolecular transfer). For example, a modulation agent may receive electromagnetic energy and re-emit energy in the form of thermal energy or energy which otherwise contributes to heating the environment in vicinity of the light emission.

In various embodiments, the energy modulation agent (down converters, mixtures of down converters, up converters, mixtures of up converters, and combinations thereof) receives energy from a source and re-emits the energy (e.g. UV-A and/or visible light). Some energy modulation agents may have a very short energy retention time (on the order of femtoseconds (fs), e.g. fluorescent molecules) whereas others may have a very long half-life (on the order of minutes to hours, e.g. luminescent or phosphorescent molecules). Suitable energy modulation agents include, but are not limited to, a biocompatible fluorescing metal nanoparticle, fluorescing dye molecules, gold nanoparticle, a quantum dot, a quantum dot encapsulated by polyamidoamine dendrimers, a luciferase, a biocompatible phosphorescent molecule, a combined electromagnetic energy harvester molecule, and a lanthanide chelate capable of intense luminescence. These energy modulation agents (some of which are described above as nanoparticles) need not be of nanometer size and can in various embodiments of this invention be of micron-sized proportions. Typically, the energy modulation agents (down converters, mixtures of down converters, up converters, mixtures of up converters, and combinations thereof) induce photoreactive changes in the medium and are not used for the purpose of exclusively heating the medium.

Within the context of the present invention, the energy modulation agent is preferably in the form of a particulate, more preferably in the form of a powder, for ease of coating the particles of the energy modulation agent.

Other suitable energy modulation agents include organic fluorescent molecules or inorganic particles capable of fluorescence and/or phosphorescence having crystalline. polycrystalline or amorphous micro-structures.

Organic fluorescent compounds with high quantum yield include, but are not limited to, naphthalene, pyrene, perylene, anthracene, phenanthrene, p-terphenyl, p-quaterphenyl, trans-stilbene, tetraphenylbutadiene, distyrylbenzene. 2,5-diphenyloxazole, 4-methyl-7-diethylaminocoumarin, 2-phenyl-5-(4-biphenyl)-1,3,4-oxadiazole, 3-phenylcarbostyryl, 1,3,5-triphenyl-2-pyrazoline, 1,8-naphthoylene-1′,2′-benzimidazole, 4-amino-n-phenyl-naphthalimide.

Inorganic fluorescent and/or phosphorescent materials span a wide variety of materials. Furthermore, these materials can be doped with specific ions (activators or a combination of activators) that occupy a site in the lattice structure in the case of crystalline or polycrystalline materials and could occupy a network forming site or a bridging and/or non-bridging site in amorphous materials. These compounds include, but are not limited to, (not ranked by order of preference or utility): CaF₂, ZnF₂, KMgF₃, ZnGa₂O₄, ZnAl₂O₄, Zn₂SiO₄, Zn₂GeO₄, Ca₅(PO₄)₃F, Sr₅(PO₄)₃F, CaSiO₃, MgSiO₃, ZnS, MgGa₂O₄, LaAl₁₁O₁₈, Zn₂SiO₄, Ca₅(PO₄)₃F, Mg₄Ta₂O₉, CaF₂, LiAl₅O₈, LiAlO₂, CaPO₃, AlF₃, and LuPO₄:Pr³⁺. Examples further include the alkali earth chalcogenide phosphors which are in turn exemplified by the following non-inclusive list: MgS:Eu³⁺, CaS:Mn²⁺, CaS:Cu, CaS:Sb, CaS:Ce³⁺, CaS:Eu²⁺, CaS:Eu²⁺Ce³⁺, CaS:Sm³⁺, CaS:Pb²⁺, CaO:Mn²⁺, CaO:Pb²⁺, Ca₃(PO₄)₂:Tl⁺, (Ca, Zn)₃(PO₄)₂:Tl⁺.

Further examples include the ZnS type phosphors that encompass various derivatives: ZnS:Cu,Al(Cl), ZnS:Cl(Al), ZnS:Cu,I(Cl), ZnS:Cu, ZnS:Cu,In.

Also included are the compound IIIb-Vb phosphors which include the group IIIb and Vb elements of the periodic table. These semiconductors include BN, BP, BSb, AlN, AlP, AlAs, AlSb, GaN, GaP, GaAs, GaSb, InN, InP, InAs, InSb and these materials may include donors and acceptors that work together to induce light emission diodes. These donors include, but are not limited to, Li, Sn, Si, Li, Te, Se, S, O and acceptors include, but are not limited to, C, Be, Mg, Zn, Cd, Si, Ge. Further included are the major GaP light emitting diodes which include, but are not limited to, GaP:Zn,O, GaP:NN, Gap:N and GaP, which emit colors Red, Yellow, Green and Pure Green respectively.

The energy modulation agents can further include such materials as GaAs with compositional variation of the following sort: In^{1-y}(Ga^{1-x}Al^{x})_{y}P.

Also included is silicon carbide SiC, which has commercial relevancy as a luminescent platform in blue light emitting diodes. These include the polytypes 3C—SiC, 6H—SiC, 4H—SiC with donors such as N and Al and acceptors such as Ga and B.

Further examples include multiband luminescent materials include, but not limited to, the following compositions (Sr,Ca,Ba)₅(PO₄)₃Cl:Eu²⁺, BaMg₂Al₁₆O₂₇:Eu²⁺, CeMgAl₁₁O₁₉:Ce³⁺:Tb³⁺, LaPO₄:Ce³⁺:Tb³⁺, GdMgB₅O₁₀:Ce₃:Tb³⁺, Y₂O₃:Eu³⁺, (Ba,Ca,Mg)₅(PO₄)₃Cl:Eu²⁺, 2SrO_{0.84}P2O50.16B2O3:Eu²⁺, Sr₄Al₁₄O₂₅:Eu²⁺.

Materials typically used for fluorescent high pressure mercury discharge lamps are also included. These can be excited with X-Ray and are exemplified by way of family designation as follows: Phosphates (Sr, M)(PO₄)₂:Sn²⁺, Mg or Zn activator, Germanate 4MgO·GeO₂:Mn⁴⁺, 4(MgO, MgF₂)GeO₂:Mn⁴⁺, Yttrate Y₂O₃:Eu³⁺, Vanadate YVO₄:Eu³⁺, Y(P,V)O₄:Eu³⁺, Y(P,V)O₄:In⁺, Halo-Silicate Sr₂Si:O₈₂SrCl₂:Eu²⁺, Aluminate (Ba,Mg)₂Al₁₆O₂₄:Eu²⁺, (Ba,Mg)₂Al₁₆O₂₄:Eu²⁺,Mn²⁺, Y₂O₃Al₂O₃:Tb³⁺.

Another grouping by host compound includes chemical compositions in the halophosphates phosphors, phosphate phosphors, silicate phosphors, aluminate phosphors, borate phosphors, tungstate phosphors, and other phosphors. The halophosphates include, but are not limited to: 3Ca₃(PO₄)₂:Ca(F,Cl)₂:Sb³⁺, 3Ca₃(PO₄)_{2.}Ca (F,Cl)₂:Sb³⁺/Mn²⁺, Sr₁₀(PO₄)₆Cl₂:Eu²⁺, (Sr,Ca)₁₀(PO₄)₆Cl₂:Eu²⁺, (Sr,Ca)₁₀(PO₄)₆·nB₂O₃:Eu³⁺, (Sr, Ca,Mg)₁₀(PO₄)₆Cl₂:Eu²⁺. The phosphate phosphors include, but are not limited to: Sr₂P₂O₇:Sn²⁺, (Sr,Mg)₃(PO₄)₂:Sn²⁺, Ca₃(PO₄)₂·Sn²⁺, Ca₃(PO₄)₂:Tl⁺, (Ca,Zn)₃(PO₄)₂:Tl⁺, Sr₂P₂O₇:Eu²⁺, SrMgP₂O₇:Eu²⁺, Sr₃(PO₄)₂:Eu²⁺, LaPO₄:Ce³⁺, Tb³⁺, La₂O₃·0.2SiO₂·0.9P₂O₅:Ce³⁺·Tb³⁺, BaO·TiO₂·P₂O₅. The silicate phosphors Zn₂SiO₄:Mn²⁺, CaSiO₃:Pb²⁺/Mn²⁺, (Ba, Sr, Mg)₃Si₂O₇:Pb²⁺, BaSi₂O₅:Pb²⁺, Sr₂Si₃O₈·2SrCl₂:Eu²⁺, Ba₃MgSi₂O₈:Eu²⁺, (Sr, Ba)Al₂Si₂O₈:Eu²⁺.

The aluminate phosphors include, but are not limited to: LiAlO₂:Fe³⁺, BaAl₈O₁₃:Eu²⁺, BaMg₂Al₁₆O₂₇:Eu²⁺, BaMg₂Al₁₆O₂₇:Eu²⁺/Mn²⁺, Sr₄Al₁₄O₂₅:Eu²⁺, CeMgAl₁₁O₁₉:Ce³⁺/Tb³⁺.

The borate phosphors include: Cd₂B₂O₅:Mn²⁺, SrB₄O₇F:Eu²⁺, GdMgB₅O₁₀:Ce³⁺/Tb³⁺, GdMgB₅O₁₀:Ce³⁺/Mn³⁺, GdMgB₅O₁₀:Ce³⁺/Tb³⁺/Mn²⁺.

The tungstate phosphors include, but are not limited to: CaWO₄, (Ca,Pb)WO₄, MgWO₄. Other phosphors Y₂O₃:Eu³⁺, Y(V,P)O₄:Eu²⁺, YVO₄:Dy³⁺, MgGa₂O₄:Mn²⁺, 6MgO·As₂O₅:Mn²⁺, 3.5MgO_{0.5}MgF₂·GeO₂:Mn⁴⁺.

The activators to the various doped phosphors include, but are not limited to: Tl⁺, Pb²⁺, Ce³⁺, Eu²⁺, WO₄²⁻, Sn²⁺, Sb³⁺, Mn²⁺, Tb³⁺, Eu³⁺, Mn⁴⁺, Fe³⁺. The luminescence center Tl⁺ is used with a chemical composition such as: (Ca,Zn)₃(PO₄)₂:Tl⁺, Ca₃ (PO₄)₂:Tl⁺. The luminescence center Mn²⁺is used with chemical compositions such as MgGa₂O₄:Mn²⁺, BaMg₂Al₁₆O₂₇:Eu²⁺/Mn²⁺, Zn₂SiO₄:Mn²⁺, 3Ca₃(PO₄)₂·Ca(F,Cl)₂:Sb²⁺/Mn²⁺, CaSiO₃:Pb²⁺/Mn²⁺, Cd₂B₂O₅:Mn²⁺, CdB₂O₅:Mn²⁺, GdMgB₅O₁₀:Ce³⁺/Mn²⁺, GdMgB₅O₁₀:Ce³⁺/Tb³⁺/Mn²⁺. The luminescence center Sn2+is used with chemical compositions such as: Sr₂P₂O₇:Sn²⁺, (Sr,Mg)₃(PO₄)₂:Sn²⁺. The luminescence center Eu²⁺ is used with chemical compositions such as: SrB₄O₇F:Eu²⁺, (Sr,Ba)Al₂Si₂O₈:Eu²⁺, Sr₃(PO₄)₂:Eu²⁺, Sr₂P₂O₇:Eu²⁺, Ba₃MgSi₂O₈:Eu²⁺, Sr₁₀(PO₄)₆Cl₂:Eu²⁺, BaMg₂Al₁₆O₂₇:Eu²⁺/Mn²⁺, (Sr,Ca)₁₀(PO₄)₆Cl₂:Eu²⁺. The luminescence center Pb²⁺ is used with chemical compositions such as: (Ba,Mg,Zn)₃Si₂O₇:Pb²⁺, BaSi₂O₅:Pb²⁺, (Ba,Sr)₃Si₂O₇:Pb²⁺.

The luminescence center Sb²⁺is used with chemical compositions such as: 3Ca₃(PO₄)₂·Ca(F,Cl)₂:Sb³⁺, 3Ca₃(PO₄)₂·Ca(F,Cl)₂:Sb³⁺/Mn²⁺.

The luminescence center Tb³⁺ is used with chemical compositions such as: CeMgAl₁₁O₁₉:Ce³⁺/Tb³⁺, LaPO₄:Ce³⁺/Tb³⁺, Y₂SiO₅:Ce³⁺/Tb³⁺, GdMgB₅O₁₀:Ce³⁺/Tb³⁺. The luminescence center Eu³⁺ is used with chemical compositions such as: Y₂O₃:Eu³⁺, Y(V,P)O₄:Eu³⁺. The luminescence center Dy³⁺ is used with chemical compositions such as: YVO₄:Dy³⁺. The luminescence center Fe³⁺ is used with chemical compositions such as: LiAlO₂:Fe³⁺. The luminescence center Mn⁴⁺ is used with chemical compositions such as: 6MgO·As₂O₅:Mn⁴⁺, 3.5MgO0.5MgF₂·GeO₂:Mn⁴⁺. The luminescence center Ce³⁺ is used with chemical compositions such as: Ca₂MgSi₂O₇:Ce³⁺ and Y₂SiO₅:Ce³⁺. The luminescence center WO₄²⁻ is used with chemical compositions such as: CaWO₄, (Ca,Pb)WO₄, MgWO₄. The luminescence center TiO₄⁴⁻ is used with chemical compositions such as: BaO·TiO₂·P₂O₅.

Additional phosphor chemistries of interest using X-Ray excitations include, but are not limited to, the k-edge of these phosphors. Low energy excitation can lead to intense luminescence in materials with low k-edge. Some of these chemistries and the corresponding k-edge are listed below:

| | |
|---|---|
| BaFCl:Eu²⁺ | 37.38 keV |
| BaSO₄:Eu²⁺ | 37.38 keV |
| CaWO₄ | 69.48 keV |
| Gd₂O₂S:Tb³⁺ | 50.22 keV |
| LaOBr:Tb³⁺ | 38.92 keV |
| LaOBr:Tm³⁺ | 38.92 keV |
| La₂O₂S:Tb³⁺ | 38.92 keV |
| Y₂O₂S:Tb³⁺ | 17.04 keV |
| YTaO₄ | 67.42 keV |
| YTaO₄:Nb | 67.42 keV |
| ZnS: Ag | 9.66 keV |
| (Zn, Cd)S:Ag | 9.66/26.7 keV |

These materials can be used alone or in combinations of two or more. A variety of compositions can be prepared to obtain the desired output wavelength or spectrum of wavelengths.

These energy modulation agents can be used in a wide variety of applications, including but not limited to, medical treatments using energy generated in vivo within a subject being treated, solar cells, adhesives and other resins, sterilization treatment for various materials (such as wastewater, beverages, etc). The use of energy modulation agents in such applications has been described in the following; US Published Application No. 2008/0248001; US Published Application No. 2009/0104212; US Published Application No. 2009/0294692; US Published Application No. 2010/0003316; US Published Application No. 2010/0016783; US Published Application No. 2010/0261263; US Published Application No. 2010/0266621; US Published Application No. 2011/0021970; US Published Application No. 2011/0117202; US Published Application No. 2011/0126889; US Published Application No. 2011/0129537; US Published Application No. 2011/0263920; US Published Application No. 2012/0064134; US Published Application No. 2012/0089180; US Published Application No. 2013/0102054; US Published Application No. 2013/0129757; US Published Application No. 2013/0131429; US Published Application No. 2013/0156905; US Published Application No. 2013/0171060; US Published Application No. 2013/0240758; US Published Application No. 2014/0134307; US Published Application No. 2014/0163303; US Published Application No. 2014/0166202; US Published Application No. 2014/0222117; US Published Application No. 2014/0242035; US Published Application No. 2014/0243934; US Published Application No. 2014/0272030; US Published Application No. 2014/0323946; US Published Application No. 2014/0341845; US Published Application No. 2014/0343479; US Published Application No. 2015/0182934; US Published Application No. 2015/0202294; US Published Application No. 2015/0246521; US Published Application No. 2015/0251016; US Published Application No. 2015/0265706; US Published Application No. 2015/0283392; US Published Application No. 2015/0290614; US Published Application No. 2016/0005503; US Published Application No. 2016/0067524; US Published Application No. 2016/0159065; US Published Application No. 2016/0243235; US Published Application No. 2016/0263393; US Published Application No. 2016/0325111; US Published Application No. 2016/0331731; US Published Application No. 2016/0354467; US Published Application No. 2016/0362534; US Published Application No. 2017/0027197; US Published Application No. 2017/0043178; US Published Application No. 2017/0050046; US Published Application No. 2017/0096585; US Published Application No. 2017/0113061; US Published Application No. 2017/0121472; US Published Application No. 2017/0154866; US Published Application No. 2017/0157418; US Published Application No. 2017/0162537; US Published Application No. 2017/0173350; US Published Application No. 2017/0186720; US Published Application No. 2017/0190166; US Published Application No. 2017/0196977; US Published Application No. 2017/0239489; US Published Application No. 2017/0239637; US Published Application No. 2017/0240717; US Published Application No. 2017/0258908; US Published Application No. 2017/0319868; US Published Application No. 2017/0319869; US Published Application No. 2018/0036408; US Published Application No. 2018/0154171; US Published Application No. 2018/0154178; US Published Application No. 2018/0169433; US Published Application No. 2018/0170028; US Published Application No. 2018/0269174; US Published Application No. 2018/0271121; US Published Application No. 2018/0304225; US Published Application No. 2018/0311355; US Published Application No. 2018/0317307; US Published Application No. 2018/0344850; US Published Application No. 2018/0358327; US Published Application No. 2019/0016869; US Published Application No. 2019/0022221; US Published Application No. 2019/0100680; US Published Application No. 2019/0134419; US Published Application No. 2019/0134595; US Published Application No. 2019/0134596; US Published Application No. 2019/0157234; US Published Application No. 2019/0168015; US Published Application No. 2019/0184190; US Published Application No. 2019/308030; US Published Application No. 2019/0336605; US Published Application No. 2019/0336785; US Published Application No. 2019/0336786; US Published Application No. 2019/0341364; US Published Application No. 2020/0009398; US Published Application No. 2020/0078600; US Published Application No. 2020/0079926; US Published Application No. 2020/0114164; US Published Application No. 2020/0196639; US Published Application No. 2020/0215611; US Published Application No. 2020/0222711; US Published Application No. 2020/0282056; US Published Application No. 2020/0306717; US Published Application No. 2020/0323711; US Published Application No. 2020/0365552; US Published Application No. 2020/0368547; US Published Application No. 2021/0035946; US Published Application No. 2021/0145028; US Published Application No. 2021/0253954; US Published Application No. 2022/0062419; US Published Application No. 2022/0080045; US Published Application No. 2022/0134131; US Published Application No. 2022/0146076; US Published Application No. 2022/0148997; US Published Application No. 2022/0159998; US Published Application No. 2022/0181292; US Published Application No. 2022/0184211; US Published Application No. 2022/0193441; US Published Application No. 2022/0226666; the contents of each of which are hereby incorporated by reference in their entireties.

### The Present Invention Method

In one embodiment, the present invention relates to a method for increasing emission output from an energy modulation agent, comprising:
drying the energy modulation agent in particulate form to reduce moisture level of the energy modulation agent by at least 25% to provide a dried energy modulation agent; and
coating the dried energy modulation agent with a coating having high transmissibility at a wavelength of primary emission from the energy modulation agent upon excitation.

The coating can be any desired coating for the energy modulation agent, preferably one that is inert in relation to the end use intended for the energy modulation agent. For example, for medical use, the coating is preferably a biocompatible coating, in order to protect the subject being treated, as well as avoid migration of any of the energy modulation agent composition into the patient's system. The coating can be made from a variety of materials, so long as the coating has the ability to transmit the emission wavelength(s) of the energy modulation agent being coated when the energy modulation agent is activated by an applied energy. Various optically transparent coatings are known, such as coatings formed from: inorganic materials, including, but not limited to, silica, phosphate, and silicon oxynitride; natural materials, including, but not limited to, silk, cellulose, and bacterial cells; hydrogels, including, but not limited to, agarose gel, polyethylene glycol (PEG) and derivatives thereof, and alginate; synthetic polymers, including, but not limited to, poly(L-lactic acid) (PLLA), poly(lactic acid) (PLA), polycaprolactone (PCL), and poly(lactic-co-glycolic acid) (PLGA); elastomers, including, but not limited to, polydimethylsiloxane (PDMS) and poly(octamethylene citrate)-poly(octamethylene maleate citrate) (POC-POMC); and multifunctional hybrid materials, including, but not limited to, cyclic olefin copolymer (COC), polycarbonate (PC), and conductive polyethylene (CPE). (for further details on these materials and optical properties see Nazempour, R, et al. Materials (Basel). 2018 August; 11(8): 1283, the entire contents of which are hereby incorporated by reference). Other options for the high transmissibility coating include a biocompatible layer-by-layer assembly of bovine serum albumin (BSA) and tannic acid (TA). (see Ermatov. T. et al. Optics Letters, Vol. 46, Issue 19, pp. 4828-4831 (2021), the entire contents of which are hereby incorporated by reference). Other possible coatings include optical coatings offered commercially by Evaporated Coatings, Inc (www.exaporatedcoatings.com). Still further high transmissibility coatings include coatings formed from deposition of methyltriethoxysilane/trimethoxymethylsilane. (see Mahadik, S. A., J Sol-Gel Sci Technol (2017) 81:791-796, the entire contents of which are hereby incorporated by reference). Most preferably, the coating is a diamond or diamond-like carbon (DLC) coating.

In one embodiment of the invention, the energy modulation agent particles are first coated with a biocompatible Ethyl Cellulose coating, and then overcoated with a second coating of Diamond Like Carbon (DLC).

Ethyl Cellulose (EC) is widely used in biomedical applications today, including artificial kidney membranes, coating materials for drugs, blood coagulants, additives of pharmaceutical products, blood compatible materials. EC and its derivatives have been widely used in various, personal care, food, biomedical and drug related applications. EC is not a skin sensitizer, it is not an irritant to the skin, and it is not mutagenic. EC is generally regarded as safe (GRAS), and widely used for example in food applications such flavor encapsulation, inks for making fruits and vegetables, paper and paperboard in contact with aqueous and fatty foods.

EC is also widely used for controlled release of active ingredients. The enhanced lipophilic and hydrophobic properties make it a material of choice for water resistant applications. EC is soluble in various organic solvents and can form a film on surfaces and around particles (such as phosphors). In one embodiment of this invention, ethyl cellulose is used to encapsulate the energy modulation agent particles to ensure that an added degree of protection is in place on the surface of the energy modulation agent particles. The particles are then preferably coated with a further coating of diamond or diamond-like carbon (DLC). In one embodiment of this invention, EC polymers with high molecular weight for permanent encapsulation and long term biocompatibility are used to encapsulate the energy modulation agent particles. In a preferred embodiment, the EC polymer can be any commercially available pharmaceutical grade ethyl cellulose polymer having sufficient molecular weight to form a coating on the energy modulation agent surface. Suitable EC polymers include, but are not limited to, the ETHOCEL brand of ethyl cellulose polymers available from Dow Chemical, preferably ETHOCEL FP grade products, most preferably ETHOCEL FP 100.

Diamond Like Carbon (DLC) films are in general dense, mechanically hard, smooth, impervious, abrasion resistant, chemically inert, and resistant to attack by both acids and bases; they have a low coefficient of friction, low wear rate, are biocompatible and thromboresistant. Tissues adhere well to carbon coated implants and sustain a durable interface. In presence of blood, a protein layer is formed which prevents the formation of blood clots at the carbon surface. For medical prostheses that contact blood (heart valves, anathomic sheets, stents, blood vessels, etc.), DLC coatings have been used.

DLC has emerged over the past decade as a versatile and useful biomaterial. It is harder than most ceramics, bio-inert, and has a low friction coefficient. DLC is one of the best materials for implantable applications. Studies of the biocompatibility of DLC demonstrate that there is no cytotoxicity and cell growth is normal on a DLC-coated surface. (DLC coatings on stainless steel have performed very well in in vitro studies of hemocompatibility. Histopathological investigations have shown good biotolerance of implants coated with the DLC. Moreover, DLC as a coating is efficient protection against corrosion. These properties make the embodiment described here with a double coating (EC and DLC) particularly advantageous for the energy modulation agent particles.

Methods for coating the energy modulation agents with EC or DLC are known to those of ordinary skill, and have been described, for example, in PCT/US2015/027058 filed Apr. 22, 2015, incorporated earlier by reference.

In one embodiment of the invention, the diamond or DLC coating is coated onto the energy modulation agent by Physical Vapor Deposition to encapsulate the energy modulation agent and to further enhance their biocompatibility.

For the DLC film, a preferred thickness is 60 to 115 nm, more preferably 60 to 90 nm, most preferably 70 nm +/−5 nm.

In the method of the present invention, the drying can be carried out in any desired manner, including, but not limited to, drying at a temperature and/or pressure sufficient to reduce the moisture content by a desired amount, or removing the moisture (or resident water) via a simple solvent exchange, such as by soaking the raw undried phosphors in one of a variety of solvents that are miscible with water, but have significantly higher vapor pressures, lower boiling points, or both.

In such a solvent exchange process, phosphors are washed in a solvent, such as one of the semi-polar alcohols (for example, methanol, ethanol, isopropanol, etc.), whereby the residual water gets replaced within the phosphor by the solvent, prior to the coating process without the need for heat or vacuum drying. Alternatively, the vapor pressure of the resident solvent (such as alcohol) then resident on the phosphor would allow more efficient and facile removal of the binary water-solvent admixture resulting in the need for lower temp, time and reduced pressure.

In drying the phosphors without the use of such solvent exchange (or even with such use of solvent exchange), the time, temperature, and pressure for drying the energy modulation are chosen such that the moisture content of the energy modulation agent is reduced during drying by at least 25 wt %, at least 40%, at least 50%, at least 60%, at least 70%, at least 75%, at least 80%, at least 90%, at least 95%, at least 98%, or at least 99%.

In one embodiment of the present invention, the drying is performed at a temperature of from 60° C. to 200° C., preferably from 90° C. to 150° C., more preferably from 100° C. to 130° C., still more preferably from 120° C. to 130° C., most preferably at a temperature of 125° C. +/−2° C.

In a further embodiment of the present invention, the pressure of drying can be any desired pressure, preferably from atmospheric pressure (760 mm Hg abs) to <1 mm Hg abs (high vacuum), more preferably from atmospheric pressure (760 mm Hg abs) to 680 mm Hg abs (low vacuum).

The drying is performed for any time period depending on the choice of temperature and pressure, and is preferably from 1 hour to 21 days, more preferably from 1-21 days, still more preferably from 3-15 days, most preferably for 10-15 days.

The inventors have found that by reducing the moisture level within the energy modulation agent particles just prior to coating with the high transmissibility coating, the energy modulation agent has higher emission output compared to the same material coated without the drying step or with insufficient drying or coated after the dried particles have been stored for a period of time. When the drying step is combined with a reduction in thickness of the coating layer, particularly for DLC coatings, significant improvements in emission output are particularly obtained using the present invention method. The emission output can be increased using the drying step or a combination of drying and using thinner coatings, with the emission output increasing by at least 25%, at least 40%, at least 50%, at least 60%, at least 70%, at least 75%, at least 80%, at least 90%, or even greater.

In certain embodiments of the present invention method the coating is applied to a thickness of 60 nm to 115 nm, more preferably 60 to 90 nm, most preferably 70 nm +/−5 nm. This is particularly the case for the preferred embodiment of using a DLC coating.

In certain preferred embodiments of the present invention, the drying is performed at a temperature from 90° C. to 150° C, at a pressure from atmospheric pressure (760 mm Hg abs) to 650 mm Hg abs for a period of time from 1-21 days. In further preferred embodiments of the present invention, the drying is performed at a temperature of 100° C. to 130° C. at a pressure from atmospheric pressure (760 mm Hg abs) to 680 mm Hg abs for a period of time from 3-15 days.

In the present invention method, the energy modulation agent can be a single compound or a mixture of two or more energy modulation agents, selected to provide desired predominant emission wavelengths. The energy modulation agent can be any of those energy modulation agents noted above, and in certain preferred embodiments, is a combination of two or more energy modulation agents.

In one preferred embodiment, the energy modulation agent in the present method is an admixture of Zn₂SiO₄:Mn²⁺ and (3Ca₃(PO₄)₂Ca(F,Cl)₂: Sb³⁺, Mn²⁺) at a ratio of Zn₂SiO₄:Mn²⁺: (3Ca₃(PO₄)₂Ca(F, Cl)₂: Sb³⁺, Mn²⁺) of from 1:10 to 10:1, or from 1:5 to 5:1, or from 1:2 to 2:1, or about 1:2. The admixture is preferably in dry solid/powder form.

### EXAMPLES

### Transmission Measurement at Different Coating Thicknesses

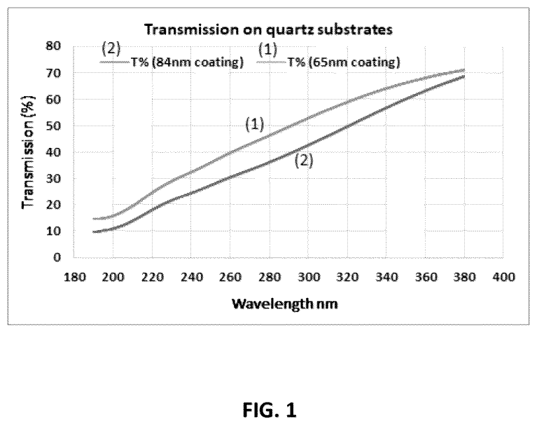

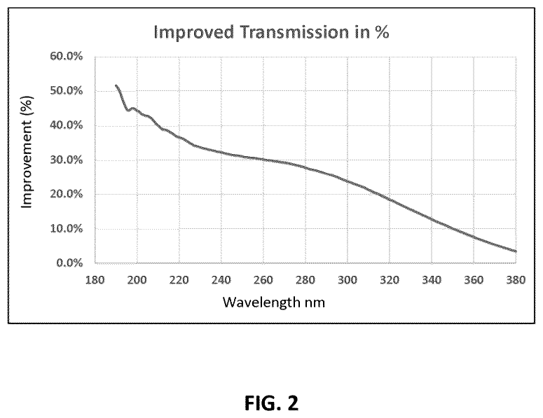

In order to show the impact of coating thickness on emission transmission, the transmission through a quartz slide that was coated with a diamond-like carbon (DLC) coating was performed. One quartz slide was coated with a DLC coating having a thickness of 65 nm+/−6 nm. A second quartz slide was coated with a DLC coating having a thickness of 84 nm+/−9 nm. The transmission at various wavelengths were measured and are shown in FIGS. 1 and 2.

FIG. 1 shows the transmission differences between the two slides over a range of wavelengths from about 190 nm to about 380 nm. FIG. 2 shows the percentage improvement in transmission at each wavelength over that range for the thinner coating (consistently higher transmission).

### Cathodoluminescence of Dried Phosphors

In order to show the improvements in emission output provided by drying the phosphors prior to coating, experiments were performed whereby one set of phosphors were dried and coated, and then the cathodoluminescense (CL) was measured and compared against the CL of a second set of phosphors that were not dried, coated, and then measured. The results are summarized in the Table below.

The two UV peaks of interest in this case were at 165 nm and 360 nm. The drying of phosphors resulted in removal of physically and chemically adhered water. As illustrated in the results below, dried phosphors resulted in significantly more light intensity measured by CL. Furthermore, the drying of phosphors prior to coating yielded more pronounced light emissions under intensity measurements such as those utilized under cathodoluminescence.

TABLE

| Processing | CPS (165 nm) | CPS (362 nm) |
|---|---|---|
| Phosphors (without drying) | 23,000 | 55,000 |
| Phosphors (with drying under vacuum) | 39,000 | 67,500 |
| % improved output under CL | 69.6% | 22.7% |
| measurements | | |

### The Combined Impact of Thinner Coating and Drying on CL Results

In one example, phosphors that were dried and coated using a DLC coating having a thickness of 65 nm+/−6 nm were compared with phosphors that were not dried, then coated with a DLC coating having a thickness of 86 nm+/−7 nm. The results under CL illustrate an surprising and significant improvement of light intensity which can drive more photo-catalytic reactions.

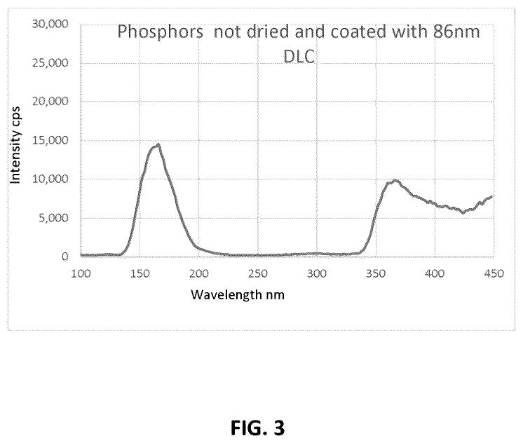

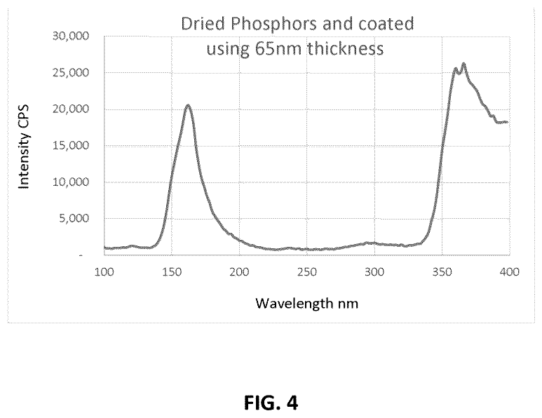

The peaks of interest in this example were 165 nm and 360 nm. The results are presented in graphical form in FIGS. 3 and 4.

FIG. 3 shows a graph of the emission intensity of the phosphors that were not dried, then coated with the 86 nm DLC coating. FIG. 4 shows a graph of the emission intensity of the phosphors that were dried then coated with the thinner 65 nm DLC coating. A dramatic increase in emission intensity was observed at both the lower emission at 165 nm and the higher higher emission at 360 nm for the phosphors of FIG. 4 that were dried then coated with the thinner DLC coating.

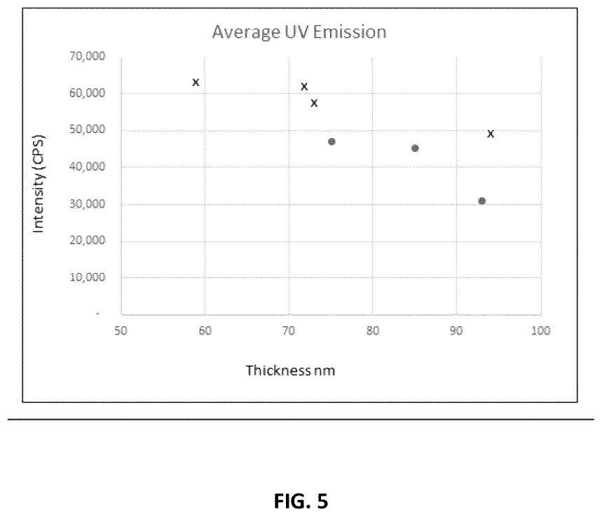

FIG. 5 shows a graph comparing the average UV emission of various phosphors coated using DLC at different thicknesses contrasted between phosphors dried under negative pressure (see data points marked “x”) and phosphors that were not dried (see data points marked “⋅”).

As is evident from the examples presented, thinner coatings result in greater emission output from the phosphors. Additionally, dried phosphors result in greater emission output compared to phosphors that were not dried, at any given coating thickness.

Accordingly, the present invention is exemplified, but not limited to, at least the following listing of embodiments:

### Embodiment 1

A method for increasing emission output from an energy modulation agent, comprising:
drying the energy modulation agent in particulate form to reduce moisture level of the energy modulation agent by at least 25% to provide a dried energy modulation agent; and
coating the dried energy modulation agent with a coating having high transmissibility at a wavelength of primary emission from the energy modulation agent upon excitation.

### Embodiment 2

The method of Embodiment 1, wherein the drying is performed at a temperature and/or pressure sufficient to reduce the moisture level.

### Embodiment 3

The method of one of Embodiments 1 or 2, wherein the coating is a diamond or diamond-like carbon (DLC) coating.

### Embodiment 4

The method of one of Embodiments 1 or 2, wherein the coating is a member selected from the group consisting of silica, phosphate, silicon oxynitride, silk, cellulose, bacterial cells, agarose gel, polyethylene glycol (PEG) and derivatives thereof, alginate, poly(L-lactic acid) (PLLA), poly(lactic acid) (PLA), polycaprolactone (PCL), poly(lactic-co-glycolic acid) (PLGA), polydimethylsiloxane (PDMS), poly(octamethylene citrate)-poly(octamethylene maleate citrate) (POC-POMC), cyclic olefin copolymer (COC), polycarbonate (PC), conductive polyethylene (CPE), a biocompatible layer-by-layer assembly of bovine serum albumin (BSA) and tannic acid (TA), and methyltriethoxysilane/trimethoxymethylsilane.

### Embodiment 5

The method of any one of Embodiments 2 to 4, wherein the temperature of drying is from 60° C, to 200° C.

### Embodiment 6

The method of any one of Embodiments 2 to 5, wherein the pressure of drying is from atmospheric pressure (760 mm Hg abs) to <1 mm Hg abs (high vacuum).

### Embodiment 7

The method of any one of Embodiments 1 to 6, wherein the coating is applied to a thickness of 60 nm to 115 nm.

### Embodiment 8

The method of Embodiment 7, wherein the coating is applied to a target setpoint thickness of 70 nm.

### Embodiment 9

The method of Embodiment 7, wherein the coating is applied to a thickness of 60 nm to 90 nm.

### Embodiment 10

The method of Embodiment 2, wherein the coating is applied to a thickness of 60 nm to 115 nm.

### Embodiment 11

The method of Embodiment 10, wherein the coating is applied to a target setpoint thickness of 70 nm.

### Embodiment 12

The method of Embodiment 10, wherein the coating is applied to a thickness of 60 nm to 90 nm.

### Embodiment 13

The method of Embodiment 4, wherein the coating is applied to a thickness of 60 nm to 115 nm.

### Embodiment 14

The method of Embodiment 13, wherein the coating is applied to a target setpoint thickness of 70 nm.

### Embodiment 15

The method of Embodiment 13, wherein the coating is applied to a thickness of 60 nm to 90 nm.

### Embodiment 16

The method of Embodiment 5, wherein the coating is applied to a thickness of 60 nm to 115 nm.

### Embodiment 17

The method of Embodiment 16, wherein the coating is applied to a target setpoint thickness of 70 nm.

### Embodiment 18

The method of Embodiment 16, wherein the coating is applied to a thickness of 60 nm to 90 nm.

### Embodiment 19

The method of Embodiment 3, wherein the coating is applied to a thickness of 60 nm to 115 nm.

### Embodiment 20

The method of Embodiment 19, wherein the coating is applied to a target setpoint thickness of 70 nm.

### Embodiment 21

The method of Embodiment 19, wherein the coating is applied to a thickness of 60 nm to 90 nm.

### Embodiment 22

The method of Embodiment 6, wherein the coating is applied to a thickness of 60 nm to 115 nm.

### Embodiment 23

The method of Embodiment 22, wherein the coating is applied to a target setpoint thickness of 70 nm.

### Embodiment 24

The method of Embodiment 22, wherein the coating is applied to a thickness of 60 nm to 90 nm.

### Embodiment 25

The method of Embodiment 2, wherein the diamond or diamond-like carbon (DLC) coating is applied to a thickness of 60 nm to 115 nm.

### Embodiment 26

The method of Embodiment 25, wherein the diamond or diamond-like carbon (DLC) coating is applied to a target setpoint thickness of 70 nm.

### Embodiment 27

The method of Embodiment 25, wherein the diamond or diamond-like carbon (DLC) coating is applied to a thickness of 60 nm to 90 nm.

### Embodiment 28

The method of Embodiment 5, wherein the coating is a diamond or diamond-like carbon (DLC) coating which is applied to a thickness of 60 nm to 115 nm.

### Embodiment 29

The method of Embodiment 28, wherein the diamond or diamond-like carbon (DLC) coating is applied to a target setpoint thickness of 70 nm.

### Embodiment 30

The method of Embodiment 28, wherein the diamond or diamond-like carbon (DLC) coating is applied to a thickness of 60 nm to 90 nm.

### Embodiment 31

The method of Embodiment 6, wherein the coating is a diamond or diamond-like carbon (DLC) coating which is applied to a thickness of 60 nm to 115 nm.

### Embodiment 32

The method of Embodiment 31, wherein the diamond or diamond-like carbon (DLC) coating is applied to a target setpoint thickness of 70 nm.

### Embodiment 33

The method of Embodiment 31, wherein the diamond or diamond-like carbon (DLC) coating is applied to a thickness of 60 nm to 90 nm.

### Embodiment 34

The method of any one of Embodiments 2 to 33, wherein the drying is performed at a temperature from 90° C, to 150° C, at a pressure from atmospheric pressure (760 mm Hg abs) to 650 mm Hg abs for a period of time from 1-21 days.

### Embodiment 35

The method of any one of Embodiments 2 to 34, wherein the drying is performed at a temperature of 100°° C, to 130°° C, at a pressure from atmospheric pressure (760) mm Hg abs) to 680 mm Hg abs for a period of time from 3-15 days.

### Embodiment 36

The method of any one of Embodiments 1 to 35, further comprising coating the dried energy modulation agent with an ethyl cellulose coating prior to coating with the diamond or diamond-like carbon (DLC) coating.

### Embodiment 37

The method of any one of Embodiments 1 to 36, wherein the energy modulation agent is a combination of two or more energy modulation agents.

### Embodiment 38

The method of any one of Embodiments 1 to 37, wherein the combination of two or more energy modulation agents is an admixture of Zn2SiO4:Mn2+and (3Ca3(PO4)2Ca(F, Cl)2: Sb3+, Mn2+) at a ratio of Zn2SiO4:Mn2+: (3Ca3(PO4)2Ca(F, Cl)2: Sb3+, Mn2+) of from 1:10 to 10:1.

### Embodiment 39

The method of any one of Embodiments 1 to 38, wherein the ratio of Zn2SiO4:Mn2+: (3Ca3(PO4)2Ca(F, Cl)2: Sb3+, Mn2+) is from 1:5 to 5:1.

### Embodiment 40

The method of any one of Embodiments 1 to 39, wherein the ratio of Zn2SiO4:Mn2+: (3Ca3(PO4)2Ca(F, Cl)2: Sb3+, Mn2+) is from 1:2 to 2:1.

### Embodiment 41

The method of any one of Embodiments 1 to 40, wherein the ratio of Zn2SiO4:Mn2+: (3Ca3(PO4)2Ca(F, Cl)2: Sb3+, Mn2+) is about 1:2.

### Embodiment 42

The method of any one of Embodiments 1 to 41, wherein the drying reduces the moisture level of the energy modulation agent by at least 40%.

### Embodiment 43

The method of any one of Embodiments 1 to 42, wherein the drying reduces the moisture level of the energy modulation agent by at least 50%.

### Embodiment 44

The method of any one of Embodiments 1 to 43, wherein the drying reduces the moisture level of the energy modulation agent by at least 60%.

### Embodiment 45

The method of any one of Embodiments 1 to 44, wherein the drying reduces the moisture level of the energy modulation agent by at least 70%.

### Embodiment 46

The method of any one of Embodiments 1 to 45, wherein the drying reduces the moisture level of the energy modulation agent by at least 75%.

### Embodiment 47

The method of any one of Embodiments 1 to 46, wherein the drying reduces the moisture level of the energy modulation agent by at least 80%.

### Embodiment 48

The method of any one of Embodiments 1 to 47, wherein the drying reduces the moisture level of the energy modulation agent by at least 90%.

### Embodiment 49

The method of any one of Embodiments 1 to 48, wherein the drying reduces the moisture level of the energy modulation agent by at least 95%.

### Embodiment 50

The method of any one of Embodiments 1 to 49, wherein the drying reduces the moisture level of the energy modulation agent by at least 98%.

### Embodiment 51

The method of any one of Embodiments 1 to 50, wherein the drying reduces the moisture level of the energy modulation agent by at least 99%.

### Embodiment 52

The method of Embodiment 1, wherein the drying is performed by solvent exchange on the energy modulation agent using a water-miscible solvent having a vapor pressure higher than water, a boiling point lower than water, or having both.

### Embodiment 53

The method of any one of Embodiments 1 to 51, further comprising, prior to drying, performing solvent exchange on the energy modulation agent using a water-miscible solvent having a vapor pressure higher than water, a boiling point lower than water, or having both.

### Embodiment 54

A coated energy modulation agent prepared by the method of any one of Embodiments 1 to 53.

### Embodiment 55

The coated energy modulation agent of Embodiment 54, wherein the coated energy modulation agent is a combination of two or more energy modulation agents.

### Embodiment 56

The coated energy modulation agent of one of Embodiments 54 or 55, wherein the two or more energy modulation agents are an admixture of Zn2SiO4:Mn2+ and (3Ca3(PO4)2Ca(F, Cl)2: Sb3+, Mn2+) at a ratio of Zn2SiO4:Mn2+: (3Ca3(PO4)2Ca(F, Cl)2: Sb3+, Mn2+) of from 1:10 to 10:1

### Embodiment 57

The coated energy modulation agent of any one of Embodiments 54 to 56, wherein the ratio of Zn2SiO4:Mn2+: (3Ca3(PO4)2Ca(F, Cl)2: Sb3+, Mn2+) is from 1:5 to 5:1.

### Embodiment 58

The coated energy modulation agent of any one of Embodiments 54 to 57, wherein the ratio of Zn2SiO4:Mn2+: (3Ca3(PO4)2Ca(F, Cl)2: Sb3+, Mn2+) is from 1:2 to 2:1.

### Embodiment 59

The coated energy modulation agent of any one of Embodiments 54 to 58, wherein the ratio of Zn2SiO4:Mn2+: (3Ca3(PO4)2Ca(F, Cl)2: Sb3+, Mn2+) is about 1:2.

Obviously, numerous modifications and variations of the present invention are possible in light of the above teachings. It is therefore to be understood that within the scope of the appended claims, the invention may be practiced otherwise than as specifically described herein.

1. A method for increasing emission output from an energy modulation agent,
comprising:
drying the energy modulation agent in particulate form to reduce moisture level of the energy modulation agent by at least 25% to provide a dried energy modulation agent; and
coating the dried energy modulation agent with a coating having high transmissibility at a wavelength of primary emission from the energy modulation agent upon excitation.

2. The method of claim 1, wherein the drying is performed at a temperature and/or pressure sufficient to reduce the moisture level.

3. The method of claim 1, wherein the coating is a diamond or diamond-like carbon (DLC) coating.

4. The method of claim 1, wherein the coating is a member selected from the group consisting of silica, phosphate, silicon oxynitride, silk, cellulose, bacterial cells, agarose gel, polyethylene glycol (PEG) and derivatives thereof, alginate, poly(L-lactic acid) (PLLA), poly(lactic acid) (PLA), polycaprolactone (PCL), poly(lactic-co-glycolic acid) (PLGA), polydimethylsiloxane (PDMS), poly(octamethylene citrate)-poly(octamethylene maleate citrate) (POC-POMC), cyclic olefin copolymer (COC), polycarbonate (PC), conductive polyethylene (CPE), a biocompatible layer-by-layer assembly of bovine serum albumin (BSA) and tannic acid (TA), and methyltriethoxysilane/trimethoxymethylsilane.

5. The method of claim 2, wherein the temperature of drying is from 60° C, to 200° C.

6. The method of claim 2, wherein the pressure of drying is from atmospheric pressure (760 mm Hg abs) to <1 mm Hg abs (high vacuum).

7. The method of claim 1, wherein the coating is applied to a thickness of 60 nm to 115 nm.

8. The method of claim 7, wherein the coating is applied to a target setpoint thickness of 70 nm.

9. The method of claim 7, wherein the coating is applied to a thickness of 60 nm to 90 nm.

10. The method of claim 2, wherein the coating is applied to a thickness of 60 nm to 115 nm.

11. The method of claim 10, wherein the coating is applied to a target setpoint thickness of 70 nm.

12. The method of claim 10, wherein the coating is applied to a thickness of 60 nm to 90 nm.

13. The method of claim 4, wherein the coating is applied to a thickness of 60 nm to 115 nm.

14. The method of claim 13, wherein the coating is applied to a target setpoint thickness of 70 nm.

15. The method of claim 13, wherein the coating is applied to a thickness of 60 nm to 90 nm.

16. The method of claim 5, wherein the coating is applied to a thickness of 60 nm to 115 nm.

17. The method of claim 16, wherein the coating is applied to a target setpoint thickness of 70 nm.

18. The method of claim 16, wherein the coating is applied to a thickness of 60 nm to 90 nm.

19. The method of claim 3, wherein the coating is applied to a thickness of 60 nm to 115 nm.

20. The method of claim 19, wherein the coating is applied to a target setpoint thickness of 70 nm.

21. The method of claim 19, wherein the coating is applied to a thickness of 60 nm to 90 nm.

22. The method of claim 6, wherein the coating is applied to a thickness of 60 nm to 115 nm.

23. The method of claim 22, wherein the coating is applied to a target setpoint thickness of 70 nm.

24. The method of claim 22, wherein the coating is applied to a thickness of 60 nm to 90 nm.

25. The method of claim 3, wherein the diamond or diamond-like carbon (DLC) coating is applied to a thickness of 60 nm to 115 nm.

26. The method of claim 25, wherein the diamond or diamond-like carbon (DLC) coating is applied to a target setpoint thickness of 70 nm.

27. The method of claim 25, wherein the diamond or diamond-like carbon (DLC) coating is applied to a thickness of 60 nm to 90 nm.

28. The method of claim 5, wherein the coating is a diamond or diamond-like carbon (DLC) coating which is applied to a thickness of 60 nm to 115 nm.

29. The method of claim 28, wherein the diamond or diamond-like carbon (DLC) coating is applied to a target setpoint thickness of 70 nm.

30. The method of claim 28, wherein the diamond or diamond-like carbon (DLC) coating is applied to a thickness of 60 nm to 90 nm.

31. The method of claim 6, wherein the coating is a diamond or diamond-like carbon (DLC) coating which is applied to a thickness of 60 nm to 115 nm.

32. The method of claim 31, wherein the diamond or diamond-like carbon (DLC) coating is applied to a target setpoint thickness of 70 nm.

33. The method of claim 31, wherein the diamond or diamond-like carbon (DLC) coating is applied to a thickness of 60 nm to 90 nm.

34. The method of claim 5, wherein the drying is performed at a temperature from 90° C. to 150° C, at a pressure from atmospheric pressure (760 mm Hg abs) to 650 mm Hg abs for a period of time from 1-21 days.

35. The method of claim 34, wherein the drying is performed at a temperature of 100° C, to 130° C, at a pressure from atmospheric pressure (760 mm Hg abs) to 680 mm Hg abs for a period of time from 3-15 days.

36. The method of claim 1, further comprising coating the dried energy modulation agent with an ethyl cellulose coating prior to coating with the diamond or diamond-like carbon (DLC) coating.

37. The method of claim 1, wherein the energy modulation agent is a combination of two or more energy modulation agents.

38. The method of claim 37, wherein the combination of two or more energy modulation agents is an admixture of Zn₂SiO₄:Mn²⁺ and (3Ca₃(PO₄)₂Ca(F, Cl)₂: Sb³⁺, Mn²⁺) at a ratio of Zn₂SiO₄:Mn²⁺: (3Ca₃(PO₄)₂Ca(F, Cl)₂: Sb³⁺, Mn²⁺) of from 1:10 to 10:1.

39. The method of claim 38, wherein the ratio of Zn₂SiO₄:Mn²⁺: (3Ca₃(PO₄)₂Ca(F, Cl)₂: Sb³⁺, Mn²⁺) is from 1:5 to 5:1.

40. The method of claim 39, wherein the ratio of Zn₂SiO₄:Mn²⁺: (3Ca₃(PO₄)₂Ca(F, Cl)₂: Sb³⁺, Mn²⁺) is from 1:2 to 2:1.

41. The method of claim 40, wherein the ratio of Zn₂SiO₄:Mn²⁺: (3Ca₃(PO₄)₂Ca(F, Cl)₂: Sb³⁺, Mn²⁺) is about 1:2.

42. The method of claim 1, wherein the drying reduces the moisture level of the energy modulation agent by at least 40%.

43. The method of claim 1, wherein the drying reduces the moisture level of the energy modulation agent by at least 50%.

44. The method of claim 1, wherein the drying reduces the moisture level of the energy modulation agent by at least 60%.

45. The method of claim 1, wherein the drying reduces the moisture level of the energy modulation agent by at least 70%.

46. The method of claim 1, wherein the drying reduces the moisture level of the energy modulation agent by at least 75%.

47. The method of claim 1, wherein the drying reduces the moisture level of the energy modulation agent by at least 80%.

48. The method of claim 1, wherein the drying reduces the moisture level of the energy modulation agent by at least 90%.

49. The method of claim 1, wherein the drying reduces the moisture level of the energy modulation agent by at least 95%.

50. The method of claim 1, wherein the drying reduces the moisture level of the energy modulation agent by at least 98%.

51. The method of claim 1, wherein the drying reduces the moisture level of the energy modulation agent by at least 99%.

52. The method of claim 1, wherein the drying is performed by solvent exchange on the energy modulation agent using a water-miscible solvent having a vapor pressure higher than water, a boiling point lower than water, or having both.

53. The method of claim 2, further comprising, prior to drying, performing solvent exchange on the energy modulation agent using a water-miscible solvent having a vapor pressure higher than water, a boiling point lower than water, or having both.

54. A coated energy modulation agent prepared by the method of claim 1.

55. The coated energy modulation agent of claim 54, wherein the coated energy modulation agent is a combination of two or more energy modulation agents.

56. The coated energy modulation agent of claim 55, wherein the two or more energy modulation agents are an admixture of Zn₂SiO₄:Mn²⁺ and (3Ca₃(PO₄)₂Ca(F, Cl)₂: Sb³⁺, Mn²⁺) at a ratio of Zn₂SiO₄:Mn²⁺: (3Ca₃(PO₄)₂Ca(F, Cl)₂: Sb³⁺, Mn²⁺) of from 1:10 to 10:1

57. The coated energy modulation agent of claim 56, wherein the ratio of Zn₂SiO₄:Mn²⁺: (3Ca₃(PO₄)₂Ca(F, Cl)₂: Sb³⁺, Mn²⁺) is from 1:5 to 5:1.

58. The coated energy modulation agent of claim 57, wherein the ratio of Zn₂SiO₄:Mn²⁺: (3Ca₃(PO₄)₂Ca(F, Cl)₂: Sb³⁺, Mn²⁺) is from 1:2 to 2:1.

59. The coated energy modulation agent of claim 58, wherein the ratio of Zn₂SiO₄:Mn²⁺: (3Ca₃(PO₄)₂Ca(F, Cl)₂: Sb³⁺, Mn²⁺) is about 1:2.
